(19) United States
(12) Patent Application Publication
(10) Pub. No.: US 2009/0124871 A1
(43) Pub. Date: May 14, 2009

(54) Tracking system

(76) Inventors: Khalil Arshak, County Limerick (IE); Francis Adepoju, Limerick (IE)

Correspondence Address: JACOBSON HOLMAN PLLC, 400 SEVENTH STREET N.W., SUITE 600, WASHINGTON, DC, 20004, US

(21) Appl. No.: 11/990,710

(22) PCT Filed: Jul. 31, 2006
(86) PCT No.: PCT/IE2006/000081

§ 371 Date: Feb. 20, 2008

(30) Foreign Application Priority Data
Aug. 22, 2005 (IE) 2005/0556

(51) Int. Cl. A61B 5/07; G08B 21/00

(52) U.S. Cl. 600/302; 340/686.1

(57) ABSTRACT

A tracking system of the invention comprises a fixed part (30) and a consumable sensor capsule (1) the location of which is tracked in real time as it moves through the GI tract. The fixed part emits (31) acoustic signals, and the capsule receives these signals and in turn generates, after a set time delay, a response which is received by the fixed part and a computation is made of the distance between the capsule and the fixed part based on the time of flight and the intervening organs as modelled in the system's processor. The response is transmitted after a pre-set time delay and so is a simulated echo. Multiple receivers (36) are located at positions on a belt (40) chosen so that interference by bone is minimised, and so that the tracking procedure is ambulatory. The capsule has sensors (12, 13) which transmit data via an RF antenna incorporated in the capsule casing.

### FIELD OF THE INVENTION

The invention relates to tracking of objects within the body (human or animal) such as consumable sensors.

### PRIOR ART DISCUSSION

It is known in the art to provide such as a tracking system, and for example US2004/0106848 describes an approach involving RF signals and a pulsed acoustic signal. A problem with the RF approach is that there is at least the perception that they can cause unintended harm to body tissue, often referred to as “thermal” effects. Although radar systems tend to make the average power emitted much lower than the peak pulse power by sending electromagnetic waves in pulses and not continuously, at high frequencies, RF signals are easily absorbed in tissue and can cause damage to tissue cells.

The quantity used to measure how much RF energy is actually absorbed in a body is called the specific absorption rate (SAR), expressed in mW/g. In the case of whole-body exposure, a standing human adult can absorb RF energy at a maximum rate when the frequency of the RF radiation is in the range of about 80 to 100 MHz, meaning that the whole-body SAR is at a maximum under these conditions (resonance). Because of this resonance phenomenon, RF safety standards are generally most restrictive for these frequencies.

The strength of a pulse of microwave radiation used in range measurements (Radars) are directional and the RF energy they generate is contained in beams that are very narrow and resemble the beam of a spotlight. RF levels away from the main beam fall off rapidly, obeying the inverse square law. The intensity I of the influence at any given radius r is the source strength S divided by the area of the sphere, I=S/(4π²).

Thus a different sensor orientation at a particular location will produce a different distance measurement.

It is also known to employ electromagnetic signals to monitor device location during colonoscopy, however the generation and monitoring or electromagnetic radiation requires complex equipment, and strict compliance with electromagnetic spectrum restrictions is required.

U.S. Pat. No. 6,120,453 describes a system in which orientation and bearing of one probe to another is determined by calculating the relative direction by which sound energy arrives at a probe.

The invention is directed towards providing a tracking system for improved tracking of objects within the body.

### SUMMARY OF THE INVENTION

According to the invention, there is provided a tracking system comprising:
an internal device configured for moving in an internal tract of the body, the internal device comprising an acoustic receiver and an acoustic transmitter, an external apparatus comprising an acoustic transmitter and a plurality of acoustic receivers,
an external controller for directing transmission of incident acoustic signals by the transmitter of the external apparatus and for monitoring detection of acoustic responses by the receivers,
an internal controller for monitoring detection of said incident signals by the receiver of the internal device and for directing transmission of said acoustic responses by the transmitter of the internal device, and
a data processor for determining time-of-flight data for the acoustic signals, and for generating location data for the internal device according to said time-of-flight data.

In one embodiment, the internal device controller directs transmission of the response after a pre-set delay from detection of the transmission, whereby the response is a simulated echo.

In one embodiment, the internal device is a capsule configured for movement in an internal tract.

In one embodiment, the external apparatus transmitter comprises a piezoelectric crystal.

In one embodiment, the internal device transmitter and receiver comprise a surface acoustic wave transducer performing both transmitter and receiver functions.

In one embodiment, the internal device transmitter generates the response signal with a pulse train of frequency different to that of a pulse train of said incident signal.

In one embodiment, the controllers ignore signals received within a time period after a first signal of a measuring point in order to eliminate reflected signals.

In one embodiment, the controllers change state to a sleep mode within said time period.

In one embodiment, the processor determines differences between times-of-flight between the internal device and the receivers and processes said data to perform the tracking computations.

In one embodiment, the external apparatus comprises a belt supporting the receivers at locations chosen to minimise interference in paths between the internal device and the receivers when the belt is worn around the patient's torso.

In another embodiment, the belt is configured to be worn and the transmitters and the receivers operate in a non-invasive manner whereby the tracking system operates in a procedure which is ambulatory.

In one embodiment, the receivers are located on the belt so that patient bone interference in the path is minimised when the belt is worn around the patient's torso.

In one embodiment, the data processor computes internal device location by re-computing a length variable at time intervals in a successive accumulation method.

In one embodiment, the variable is initialised at a reference position in a reference volume and is re-computed only while the location is with in said reference volume.

In one embodiment, the reference volume is cylindrical.

In a further embodiment, the data processor compensates for organ densities in the paths between the internal device and the external apparatus receivers.

In one embodiment, retrograde peristalsis is accommodated by the processor.

In one embodiment, the internal device comprises a capsule configured for movement in an internal tract, and the capsule comprises a sensor for internal investigation.

In one embodiment, the capsule comprises a pressure sensor for measuring internal tract pressure.

In one embodiment, the internal device comprises a casing which facilitates acoustic transmission and reception compatible with human organs.

In one embodiment, the internal device comprises a casing which operates as an RF transmitter for a sensor.

### DETAILED DESCRIPTION OF THE INVENTION

### Brief Description of the Drawings

The invention will be more clearly understood from the following description of some embodiments thereof, given by way of example only with reference to the accompanying drawings in which:

FIG. 1(a) is a diagram of a consumable sensor telemetry capsule of the invention, showing blocks of embedded sensor and tracking components;

FIG. 1(b) is a diagram of the capsule, showing its dimensions;

FIG. 2(a) is a functional block diagram of the capsule electronics;

FIG. 2(b) is a block diagram of external components of a tracking system, showing transmitter and receiver circuitry and a facility to record data captured into memory;

FIG. 3 is a diagram showing physical arrangement of the transmit and receive external components on a control belt (c-BELT);

FIG. 4(a) is a diagram showing the physiological settings for the capsule and the c-BELT, and application to the human GI tract; FIG. 4(b) is a diagram showing the anatomy of the human GI tract, including a dimension D#1 used in a location tracking algorithm, FIG. 4(c) is a diagram showing the volumetric equivalence of the GI tract as approximated to a 3-D cylindrical object, and in which dimensions D#2 and D#3 are other variables that are used in the location tracking algorithm, and FIG. 4(d) is an image showing the side and front views of a human torso, indicating the accurate position of modelling variables D#1, D#2 and D#3;

FIG. 4(e) is a diagram of a multi-path scenario between a transmitter and a receiver, in which a pulse from the transmitter is subject to reflections;

FIGS. 5, 6, 7(a) and 7(b) are diagrams illustrating geometrical methods executed by the processor to determine the capsule's location within the intestine at a given time after the capsule has been swallowed;

FIG. 8 is a diagram showing a segment of the GI tract at position Cₙ₋₁, time tᵢ₋₁ and at a new position Cₙ at time tᵢ, and in which total length of segments is successively accumulated in software into a variable after a predetermined time interval;

FIG. 9 is a sample set of signal pulses from an external transmitter (frequency f=1 Mz), echo pulse from the capsule (frequency nf≠f; e.g. 2 MHz), and received pulses at the external receivers, (also frequency nf) wherein capsule position is determined based on TOF computations; and

FIG. 10 shows plots of live acoustic pulses before and after rectification.

### DESCRIPTION OF THE EMBODIMENTS

A tracking system of the invention comprises a fixed part and a consumable sensor capsule the location of which is tracked in real time as it moves through the GI tract. The principle of operation is that the fixed part emits acoustic signals, and the capsule receives these signals and in turn generates, after a set time delay, a response which is received by the fixed part and a computation is made of the distance between the capsule and the fixed part based on the time of flight and the intervening organs as modelled in the system's processor. The response is transmitted after a pre-set time delay and so is a simulated echo.

The capsule generates a pulse train which is an acoustic pulse, having a frequency characteristic distinctive from that of the incident pulse from the external transmitter. The external receivers distinguish between such pulses and any other pulses that might arrive due to reflection, refraction or diffraction. Such pulses are used to trigger the receiver electronics in order to record their time-of-arrival for the purpose of TOF computation.

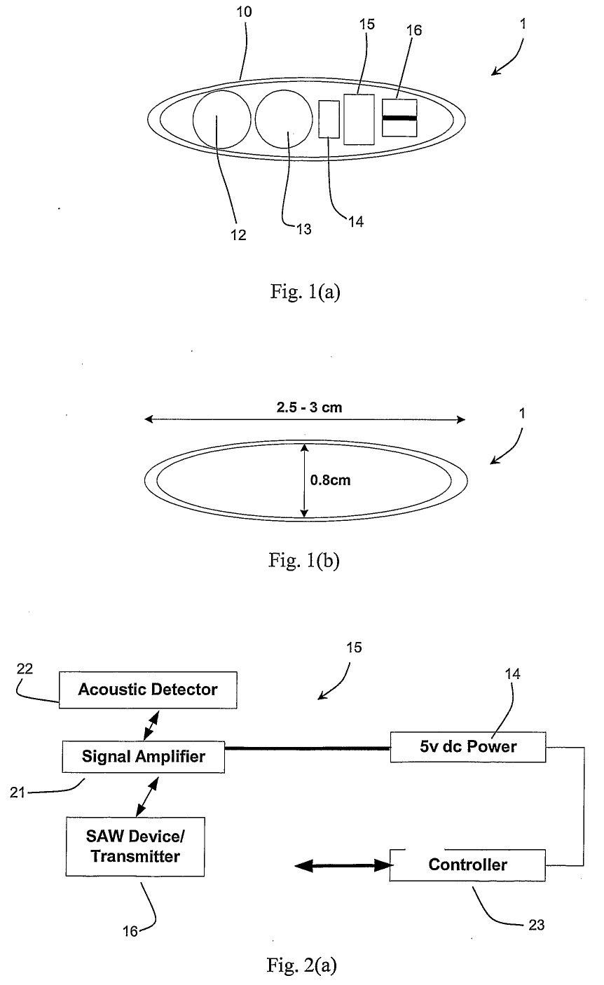

Referring to FIG. 1(a) a telemetry capsule 1 comprises:
a body 10 of polyethylene (or alternatively tempered gelatine, for example) of ellipsoidal shape coated with a suitable material (such as titanium or platinum/iridium material) that is compatible for use in the human body,
embedded temperature sensors and associated circuitry 12,
embedded pressure sensors and associated circuitry 13,
an integrated power supply unit 14, and
electronics 15 comprising a PIC microcontroller and other components for acoustic detection and transmission, and an acoustic transducer 16.

The capsule body 10 is used as an RF antenna for other applications within the capsule, such as pressure sensor circuitry, and also to help in entrapment of wave energy to help in the mechanical detection of acoustic pulses by the acoustic transducer.

The capsule 1 is sized so that it can be easily swallowed. As shown in FIG. 1(b), an average size of the ellipsoidal capsule is 2.5-3 cm in length (major axis) and 0.8 cm in width (minor axis). With increasing miniaturisation the size may be smaller.

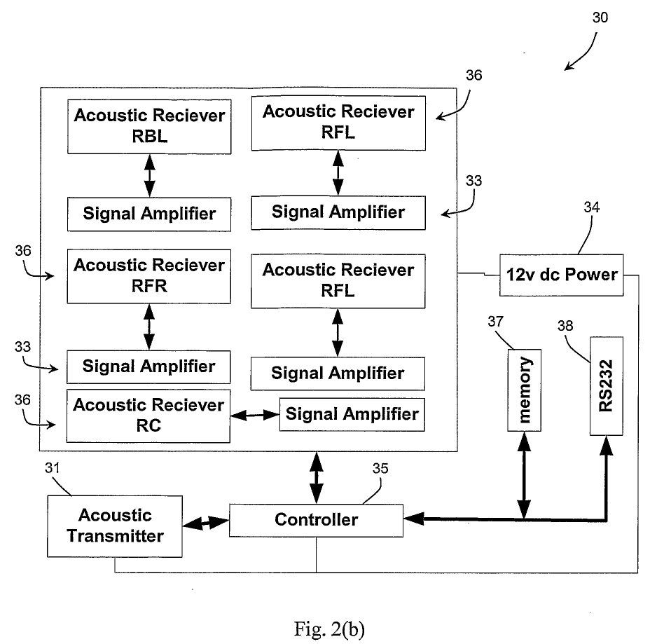

FIG. 2(a) is block diagram showing the capsule's 5V power supply 14, the surface acoustic wave (SAW) transducer 16 for transmitting and receiving acoustic pulses, and the electronics 15, including:
a signal amplifier and excitation stage 21 to produce adequate voltage to excite the transducer,
a pulse detection circuit (acoustic detector) 22, and
a microcontroller and circuitry 23 for logic, timing, interrupt, system control, and auxiliary functions.

The circuit components are fabricated using an Application-Specific Integrated Circuit (ASIC). Power is conserved within the capsule since the entire circuitry goes to sleep once the transmission of a pulse is accomplished after a prior pulse has been detected. The capsule wakes up again the next time a pulse is detected at the embedded microcontroller, usually after a pre-set time interval (e.g. 10 mins).

FIG. 2(b) is block diagram of the fixed part 30 of the position tracking system, consisting of the following elements:
an acoustic transducer 31 for transmitting incident acoustic signals and receivers 36 for receiving acoustic response signals,
a signal amplifier and excitation stage 33 to produce adequate voltage to excite the transducer 31,
12V Battery and associated regulation circuitry, 34,
a microcontroller and circuitry 35 for logic, timing, interrupt, system control, and auxiliary functions,
a pulse detection circuit 36, including band pass filters to filter the incoming pulse signals, and
peripherals such as additional memory 37 and an RS232 port 38 connected to the microprocessor to facilitate storage of intermediate data and also to communicate with a personal computer.

The transmitters preferably operate at a frequency in excess of 1 MHz in order to consistently penetrate the human tissue.

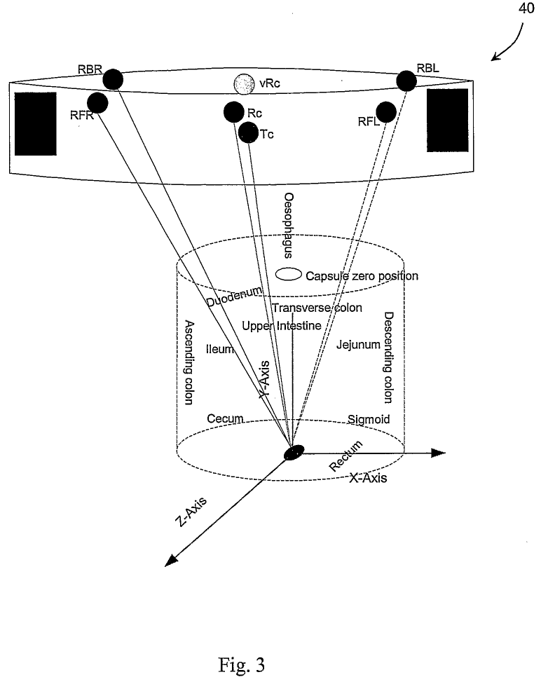

The receivers are mounted on a belt 40 at locations RBL, RFL, RFR, RFL and RC as illustrated in FIG. 3. This provides an array of transmit and receive ultrasonic sensors arranged with pre-determined geometrical spacing to facilitate computation of a “LEN” variable in order to determine the real-time location of the capsule. The controller 35 is linked with a data logger via the RS232 interface 38 to store the TOF data into memory. This data can be downloaded onto a PC for processing after the capsule has been ejected from the patient's body.

Referring again to FIG. 3, the external (c-BELT) receivers 36 include:
acoustic transmitter, centre (TC);
acoustic receiver, centre (RC);
acoustic receiver, back left (RBL);
acoustic receiver, front left (RFL);
acoustic receiver, back right (RBR);
acoustic receiver, front right (RFR).

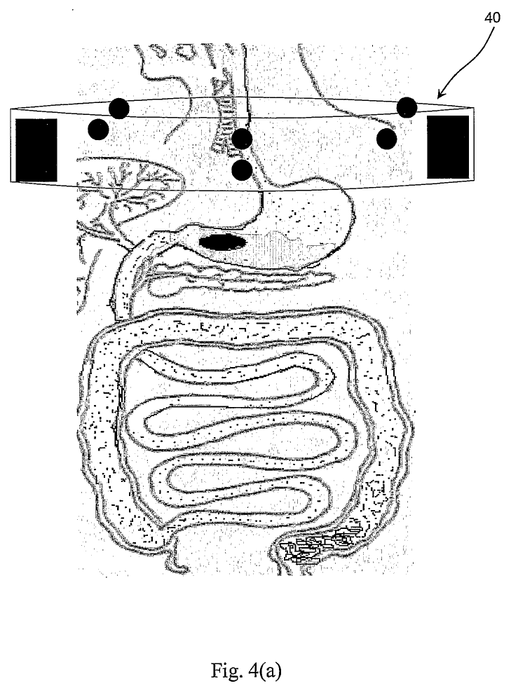

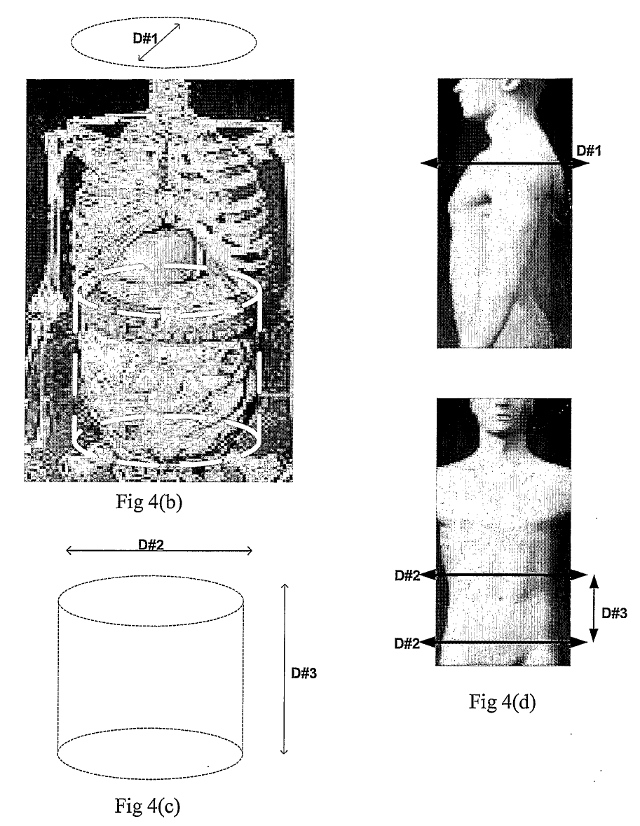

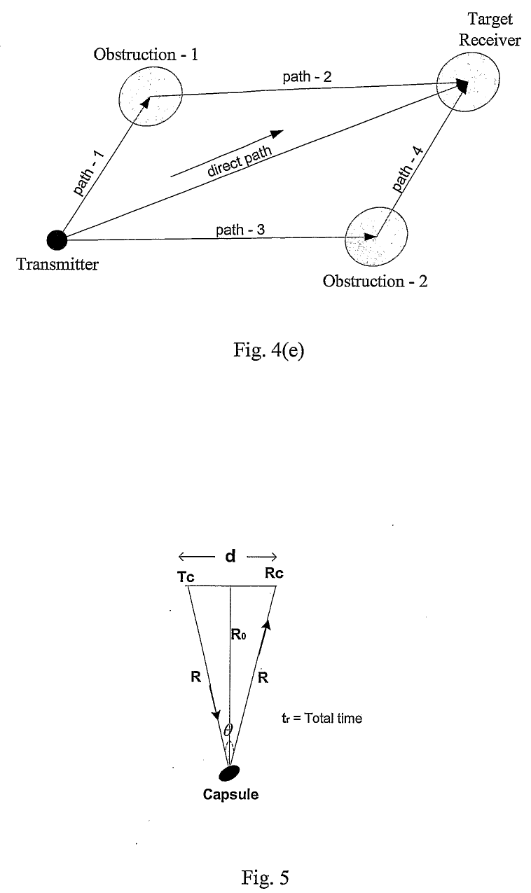

The c-Belt 40 is of leather or other suitable material (reasonable height e.g. 12 cm) that can be worn on the upper part of the torso as shown in FIG. 4(a). The circumference during test is adjustable for the patient's unique chest diameter (#D1). Transmission of pulses begins at TC, strategically placed as shown in FIG. 3, while all other receivers are positioned as shown in the same figure, taking advantage of the gaps between the ribs. Facility to compute time-of-flight is on the microprocessor residing on the c-Belt, and so also is the facility to connect to a PC via the RS232 port 38. The acoustic receivers are installed in the c-Belt 40 in such a way as to be able to couple them to the human body with a low-impedance coupler for example, propylene glycol.

As shown in FIG. 4(a), it is established that a consequential response to a natural order of contraction and expansion movement within the GI tract is antegrade movement of the capsule 1 alongside the intestinal fluid from the oesophagus to the anus, plus a few occurrences of retrograde motion along the small intestine. This motion phenomenon is generally known as peristalsis. In this invention, we take advantage of peristalsis to determine the capsule's location at any time during test.

Within the capsule 1, the embedded electronic circuits are required to produce power, timing, and vibrations within the acoustic transducer device in order to generate the pulse wave (“echo pulse”) that is transmitted to the external receivers. The real-time location of the capsule is determined geometrically by noting the round trip time of flight (TOF) of acoustic pulse signals from the acoustic transmitter, external to the capsule and, on to the receiver/transmitter inside the capsule and through to the centre, left and right external receivers, all external receivers being controlled by the external microcontroller circuit.

In operation, the capsule 1 is removed from its packaging and initialized by enabling the embedded power source. It is then swallowed with water (or any other safe liquid) by the patient. The patient switches the external control circuit ON and continues with his normal activities (ambulatory). This is one of the advantages of this invention.

At about 30 min after the capsule has been swallowed, the external transmitter is activated by the external microcontroller to send a burst of acoustic pulses. This sequence is repeated at pre-determined intervals resulting in a regular transmission of acoustic pulses from the external transmitter 31.

The capsule 1 receiving electronics 15 picks up a transmitted pulse after a short time delay and the inbuilt timing circuit activates the transmission of another acoustic pulse from the capsule 1 in response to the received pulse. This transmitted pulse is described as a reflected pulse or “echo pulse” in this invention. This reduces the undesirable effect of multi-path fading. The term “multi-path” describes a situation in which a transmitted signal follows several propagation paths from a transmitter to a receiver. As shown in FIG. 4(e), this results from the signal reflecting off several objects between the transmitter and the receiver. However, the first signal to reach the receiver is usually the one travelling undisturbed from transmitter to receiver. The others will arrive at a later time. In this invention, the first pulse train to arrive from the capsule is usually recognized by the microprocessor while those arriving at a later time have no further effect since the processor will have gone into a sleep mode.

The spatial location of the capsule is approximated by the average transmission time and the mean value of speed of sound in human tissue, which has been determined to equal 1540 ms⁻¹. As illustrated in FIG. 5, distance R between the transmitter T_{c} and the capsule 1 is determined by:

$$R = \frac{1}{2} * v * {t}_{r}$$

### Where

v=speed of wave in tissue,
R=distance from T_{c} to Capsule,
tᵣ=round trip time of acoustic wave

Let θ=angle between the incident and reflected wave.

Then by the rule of triangles,

$${R}_{0} = R * cos ( \frac{θ}{2} )$$

where
R₀ is illustrated in FIG. 5.

If the transmitter and receiver are reasonably close together, then the distance d between T_{c} and R_{c} can be approximated to zero.

Therefore, in the Limit_{θ→0},

cos(θ/2)≈cos(0)≈1.

Therefore, R₀≈R.

The initial position of the capsule is set to zero at the ‘capsule zero position’ or at the pre-duodenum region, i.e. at the pylorus. Afterwards, the capsule enters the duodenum, which is about 26 cm long.

The starting approximation is computed as follows:

Anywhere inside the duodenum, the distance covered is approximated to:

LEN=0 (length at the pylorus)+30 (approx. length of Duodenum)≈30 cm.

This is a reasonable assumption since the transit time within the length of the duodenum is relatively short.

The controller executes a successive accumulation method to track the capsule 1 inside a virtual cylinder in 3D. Due to variation in size and shape of patients, some parameters D#1, D#2, D#3 of the patient's torso will be taken in order to accurately determine the location of the capsule as modelled by a virtual cylinder shown in FIGS. 4(b) to 4(d). As shown in these drawings, the region of interest in the body can be represented by a cylindrical object with dimensions reflecting the real size/shape of the patient. The approach used to locate the capsule in 3D is as illustrated in FIGS. 5, 6, 7, and 8.

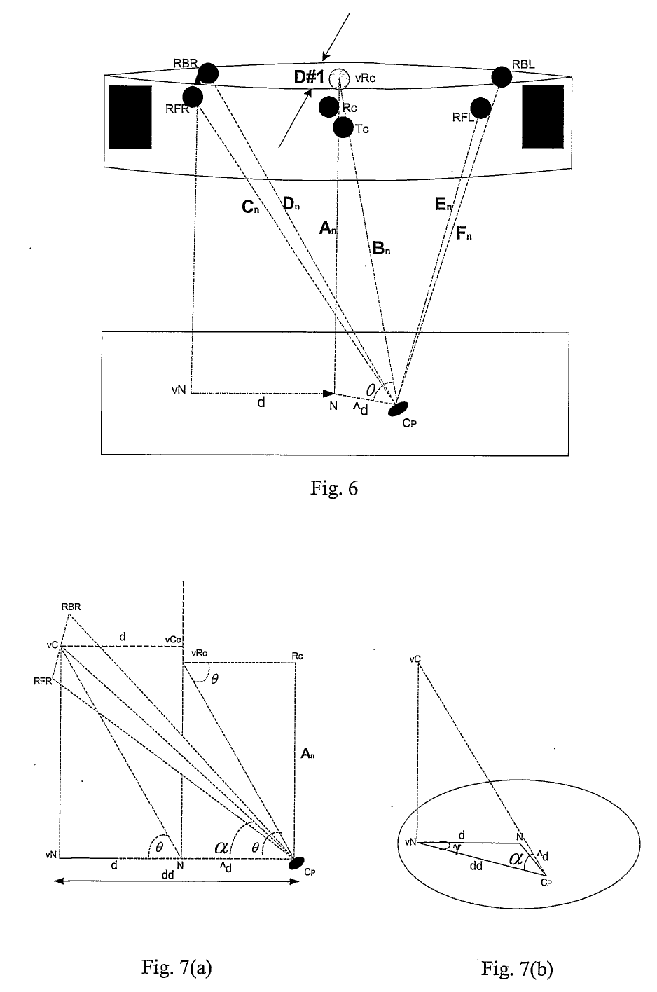

A virtual receiver vR_{c} is assumed as shown in FIG. 6 midway into D#1. This forms the Normal positional reference at the moving base of virtual cylinder which the capsule will use as a reference location in the computation of relative positions at any time tₙ. As an example, since Lengths CₚRBR and CₚRFR equates to corresponding TOF for the right back and right front positions of the external receivers, a centre of the base of a triangle formed by the two receivers with respect to the capsule at Cₚ is approximately midway between the two external receivers (designated vC).

Once the initial zero position at the top of the virtual cylinder is determined as shown above, subsequent motion of the capsule results in an increase of path traversed and the virtual cylinder grows downwards. This is reflected in the non-uniform increasing or decreasing value of program variable LEN as shown in the pseudo code below.

During the next 30 min, a pulse is transmitted from the external transmitter and consequently reflected back from the capsule and the TOF is used to compute Aₙ, Bₙ, Cₙ, Dₙ, Eₙ, Fₙ. as shown in FIG. 6.

Assuming that the receivers are not too far from vR_{c}, and are symmetrically spaced about the centre of the c-BELT, then (RFRRBR)/2≈(D#1)/2 (≈vC).

A Normal (vCvN) drawn at this point will make an angle α with a virtual line coming from the capsule at Cₚ. To determine the value of at for any capsule position Cₚ₍ₙ₎ a reference to FIG. 7(b) shows that for a triangle vNNCₚ, if angle γ is small (as is the case), then cos(γ)≈1. Therefore we can safely assume that d=dd (i.e. NCp→0).

With dd and vCCₚ mown, the value of αₙ can be determined, ie. dd=vCCp cos(α).

Which equates to

$$α = {cos}^{- 1} ( \frac{d}{{vCC}_{p}} ) .$$

To determine the length (depth) of virtual cylinder given by Aₙ, vCvN is computed from the value of α and vCCp. From FIG. 7(a), angle θ can be computed as:

$$θ = {tan}^{- 1} ( \frac{{vC}_{v} N}{d} ) .$$

Then the value of Δd (NCp) is obtained as: Δd=vR_{c}Cₚ cos(θ).

Aₙ gives the depth of the capsule relative to the capsule zero position at the virtual centre, while ((dd)²+(Δd)²)^{1/2} gives the location of the capsule from either sides of the cylinder.

This procedure will be performed for all future locations of the capsule, starting from when the virtual cylinder has zero height onto the maximum permitted height, D#3.

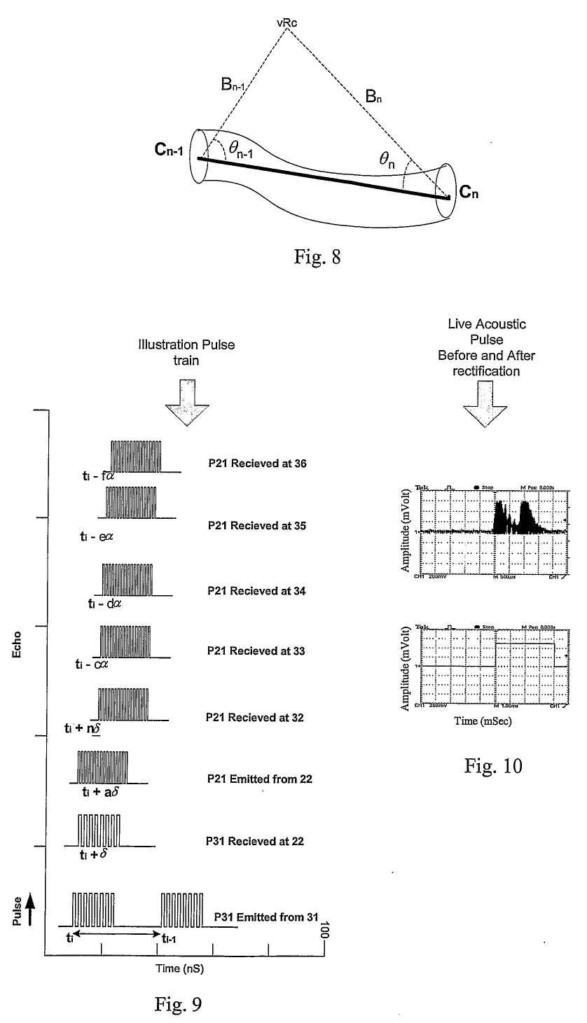

To determine the length of intestines covered, as shown in FIG. 8, the new length traversed by the capsule simply adds up from what it was at time tᵢ₋₁ with the new value of Cₙ₋₁Cₙ at tᵢ. This can be computed as follows by noting the angles θₙ computed earlier at time tₙ and the distance between the capsule and the virtual central receiver:

$$Segment length LEN = {\sum }_{n = 0}^{{n}_{max}} ( LEN )$$

$$0 \leq n \leq {n}_{max} ; 0 \leq t \leq Max\_hrs ;$$

Similarly, the above argument holds for the set of receivers RBL and RFL. Suffice to mention that when the “echo pulse” returns to the five external receivers, times of arrival are stored in memory and upon download to the PC, a comparison is made first to determine which sets of data are smaller between RFR, RFL and also between RBR and RBL. The smaller of the sets will be used to compute the capsule's location since this sort of comparison can be utilized to quickly confirm the section of the body where the capsule is situated.

The following is a list of ‘C++’ based pseudo code used to compute and render the location of the capsule after all data have been uploaded to a PC:

Accept input variables D#1, D#2, D#3;
Download data from External controller interface;
void main(int D#1, int D#2, int D#3) {

Initialise the value of LEN to zero at centre top of Cylinder;
Compute First segment approximation of the Length of the
Duodenum;
Store this value into LEN variable;
While (capsule lies within the virtual cylinder) {

Compare relevant values of input data;
Determine that capsule is within the right or Left hand

side of the body;

Compute the capsules location;
Compensate for path loss and other factors;
Add the computed length to LEN and store value back to

LEN;

```
}
If (algorithm detects that the capsule persistently
  lies outside the boundary of the Cylindrical model
  OR if after n days equivalent of data) {
```

Stop computing;
Write location data to file;
Close any remaining files;
End;

}

}

In order to compensate for path loss, the conductivity and permittivity of the tissues of the organs present in the lower region of the human body are taken into account, as shown in Table 1. A simple saline solution to simulate the electrical characteristics of the human tissue was made by altering the conductivity and permittivity of water using analytical-grade NaCl and distilled water mixed at the ANSI recommended ratio of 1.8 g/l or 0.18% NaCl at 21° C. To make adequate volume to simulate the average capacity of the stomach (1500 ml) for our experiments, 30.6 g of salt were added to 1700 ml of distilled water (17 litres×1.8 g/litre=30.6 g). The above algorithm takes care of this aggregation in full.

TABLE 1
Conductivity (siemens/cm) and Permittivity of some Tissues

| Tissue | Conductivity(S/cm) | Permittivity |
|---|---|---|
| Small intestine | 128.09 | 1.74 |
| Kidney | 117.43 | 0.89 |
| Gall bladder | 104.62 | 1.39 |

The above data is used by the fixed controller 35 in computation of capsule position.

A pulse train is illustrated in FIG. 9. Referring to FIG. 10, this is as result of a successful transmit and reflection of a pulse. The “echo pulse” was obtained with Tektronix 60 MHz TDS 200-Series Digital Real-Time Oscilloscope. (Composition from ASCII to graph was done with GSView 4.6). The top figure is the analogue while the lower figure represents the rectified digital equivalent (2.5 mV at 1 ms per division). Any rectified pulse below 2.5 mV is generally considered as a noise input.

The tracking processing may be performed by a local processor on the belt or by a linked host computer. Host computer processing is particularly useful where location data to a fine tolerance is required, giving rise to intensive processing.

Irrespective of where the tracking processing is performed, a human readable version of data is generated and displayed on a computer screen for the physician to visually locate a particular section of the intestine based on real-time computed length of intestines.

It will be appreciated that object tracking is realised in real-time, and is particularly advantageous for monitoring conditions such as Irritable Bowel Syndrome (IBS). The physician does not need to use invasive endoscopy to investigate abnormality in the GI tract.

The capsule may be arranged to include a range of sensors for capturing data which is advantageously coupled with the tracked 3D location data, as required for medical investigations and procedures.

The invention is not limited to the embodiments described but may be varied in construction and detail.

1-21. (canceled)

22. A tracking system comprising:
an internal device configured for moving in an internal tract of the body, the internal device comprising an acoustic receiver and an acoustic transmitter,
an external apparatus comprising an acoustic transmitter and a plurality of acoustic receivers,
an external controller for directing transmission of incident acoustic signals by the transmitter of the external apparatus and for monitoring detection of acoustic responses by the receivers,
an internal controller for monitoring detection of said incident signals by the receiver of the internal device and for directing transmission of said acoustic responses by the transmitter of the internal device, and
a data processor for determining time-of-flight data for the acoustic signals, and for generating location data for the internal device according to said time-of-flight data.

23. The tracking system as claimed in claim 22, wherein the internal device controller directs transmission of the response after a pre-set delay from detection of the transmission, whereby the response is a simulated echo.

24. The tracking system as claimed in claim 22, wherein the internal device is a capsule configured for movement in an internal tract.

25. The tracking system as claimed in claim 22, wherein the external apparatus transmitter comprises a piezoelectric crystal.

26. The tracking system as claimed in claim 22, wherein the internal device transmitter and receiver comprise a surface acoustic wave transducer performing both transmitter and receiver functions.

27. The tracking system as claimed in claim 22, wherein the internal device transmitter generates the response signal with a pulse train of frequency different to that of a pulse train of said incident signal.

28. The tracking system as claimed in claim 22, wherein the controllers ignore signals received within a time period after a first signal of a measuring point in order to eliminate reflected signals.

29. The tracking system as claimed in claim 28, wherein the controllers change state to a sleep mode within said time period.

30. The tracking system as claimed in claim 22, wherein the processor determines differences between times-of-flight between the internal device and the receivers and processes said data to perform the tracking computations.

31. The tracking system as claimed in claim 22, wherein the external apparatus comprises a belt supporting the receivers at locations chosen to minimise interference in paths between the internal device and the receivers when the belt is worn around the patient's torso.

32. The tracking system as claimed in claim 22, whereby the belt is configured to be worn and the transmitters and the receivers operate in a non-invasive manner whereby the tracking system operates in a procedure which is ambulatory.

33. The tracking system as claimed in claim 31, wherein the receivers are located on the belt so that patient bone interference in the path is minimised when the belt is worn around the patient's torso.

34. The tracking system as claimed in claim 22, wherein the data processor computes internal device location by re-computing a length variable at time intervals in a successive accumulation method.

35. The tracking system as claimed in claim 34, wherein the variable is initialised at a reference position in a reference volume and is re-computed only while the location is with in said reference volume.

36. The tracking system as claimed in claim 34, wherein the variable is initialised at a reference position in a reference volume and is re-computed only while the location is with in said reference volume wherein the reference volume is cylindrical.

37. The tracking system as claimed in claim 22, wherein the data processor compensates for organ densities in the paths between the internal device and the external apparatus receivers.
paths between the internal device and the external apparatus receivers.

38. The tracking system as claimed in claim 22, wherein retrograde peristalsis is accommodated by the processor.

39. The tracking system as claimed in claim 22, wherein the internal device comprises a capsule configured for movement in an internal tract, and the capsule comprises a sensor for internal investigation.

40. The tracking system as claimed in claim 39, wherein the capsule comprises a pressure sensor for measuring internal tract pressure.

41. The tracking system as claimed in claim 22, wherein the internal device comprises a casing which facilitates acoustic transmission and reception compatible with human organs.

42. The tracking system as claimed in claim 39, wherein the internal device comprises a casing which operates as an RF transmitter for a sensor.
